(19) United States
(12) Patent Application Publication
(10) Pub. No.: US 2013/0216971 A1
(43) Pub. Date: Aug. 22, 2013

(54) WIRELESS INTRA-ORAL IMAGING SYSTEM AND METHOD

(71) Applicant: I Practice Marketing, LLC, (US)

(72) Inventor: Joel Friddell, Nashville, TN (US)

(73) Assignee: I PRACTICE MARKETING, LLC, Hermitage, TN (US)

(21) Appl. No.: 13/772,846

(22) Filed: Feb. 21, 2013

Related U.S. Application Data
(60) Provisional application No. 61/601,166, filed on Feb. 21, 2012.

(51) Int. Cl. A61B 5/00

(52) U.S. Cl. CPC A61B 5/0088; A61B 5/0013; A61B 5/742
USPC 433/29; 433/215

(57) ABSTRACT

An intra-oral imaging system includes a handheld intra-oral camera in wireless communication with a remote computing device. The computing device includes a display unit and is linked to databases containing historical data for the teeth of a subject. A dentist actuates a touch-sensitive button to prompt capture of images of the subject's teeth, which are wirelessly transmitted to the computing device and graphically displayed. The images generally represent an arrangement and conditions for the teeth. Program modules are executable to identify a current condition of one or more teeth, and to select a prerecorded visual demonstration of a dental procedure from a database based on the identified current condition. Alternatively, the program modules may extrapolate using the current and historical data to predict a future condition and recommend procedures or otherwise alert the subject to potential future procedures. Visual demonstration of the procedures is provided on the display unit.

### CROSS-REFERENCES TO RELATED APPLICATIONS

This application claims benefit of the following patent application which is hereby incorporated by reference: U.S. Provisional Patent Application No. 61/601,166, filed Feb. 21, 2012.

A portion of the disclosure of this patent document contains material that is subject to copyright protection. The copyright owner has no objection to the reproduction of the patent document or the patent disclosure, as it appears in the U.S. Patent and Trademark Office patent file or records, but otherwise reserves all copyright rights whatsoever.

### BACKGROUND OF THE INVENTION

The present invention relates generally to an intra-oral imaging system and associated methods. More particularly, this invention relates to a system including an intra-oral camera communicatively coupled to a portable computing device such as for example a tablet computer or smart phone, and effective thereby to present, reference and demonstrate dental images and procedural demonstrations in substantially real-time to an associated patient.

There are presently two other types of intra-oral cameras: tethered and wireless.

The tethered camera is historically the most popular. This type of camera has to be physically connected to a computer by USB port or by some type of docking station.

The wireless cameras transport pictures using wireless radio frequencies connecting to personal computers using proprietary software. They also require some type of personal computer connection or external memory component.

The problem is that there is no existing intra-oral camera that works with mobile devices, such as tablets or smart phones, so that pictures and video can be easily transported between the two. Presently, a dentist can transport pictures from an intra-oral camera to these mobile devices, but it must be accomplished through the use of other computers and/or external memory components. The present process requires several steps and is quite cumbersome since there are no common external connections (example: USB port) on mobile devices.

Another problem that exists in the art is that there is no camera or system available that directly and seamlessly couples an intra-oral camera with a patient education and/or demonstration program.

### BRIEF SUMMARY OF THE INVENTION

In accordance with one aspect of the present invention, an intra-oral camera system is provided that is able to transport these files seamlessly and wirelessly to the mobile device by using proprietary application software (apps) coupled with Bluetooth technology.

In accordance with another aspect of the present invention, after seeing a picture of their own tooth problem in substantially real-time, the patient can immediately view similar examples and solutions from a menu of dental cases, videos and patient interviews, thus conveying complete information about possible solutions and outcomes to their problem. This patient education system is a software program that exists within the intra-oral camera app itself.

In various embodiments, the intra-oral system and methods as described herein may alternatively be applied in various other applications within the scope of the present invention, such as for example as an intra-cavity system for medical, dental, or veterinary applications. Another application may be for non-cavity healthcare related imaging applications, such as for example dermatological or other skin imaging examples. Still another example may for even non-patient related applications such as for example mechanical equipment imaging by or for service personnel, as may be used for HVAC systems or the like.

In a particular embodiment of the present invention, an intra-oral imaging system includes a handheld intra-oral camera in wireless communication with a remote computing device. The computing device includes a display unit and is linked to databases containing historical data for the teeth of a subject. A dentist actuates a touch-sensitive button to prompt capture of images of the subject's teeth, which are wirelessly transmitted to the computing device and graphically displayed. The images generally represent an arrangement and conditions for the teeth. Program modules are executable to identify a current condition of one or more teeth, and to select a prerecorded visual demonstration of a dental procedure from a database based on the identified current condition.

Alternatively, the program modules may extrapolate using the current and historical data to predict a future condition and recommend procedures or otherwise alert the subject to potential future procedures.

Visual demonstration of the procedures is provided on the display unit. In one example, a first portion of the display unit may be dedicated as a first interface wherein current, historical and/or extrapolated future images are displayed. A second portion of the display unit may be dedicated as a second interface wherein a prerecorded demonstration of a current, recommended or potential future dental procedure may be displayed.

In one example, the touch-sensitive button is effective to generate a plurality of images (e.g., 3-5 images) with a single user actuation. The button may be configured to actuate without requiring pressure or a push by the user, wherein images may be captured in a steady, clear, blur-free manner.

In another example, an imaging device including the intra-oral camera may further include movement sensors such as angular velocity sensors for further providing feedback to a vibration sensitivity program engine that in conjunction with the touch-sensitive button further allows for steady image capture.

In yet another example, user actuation may be provided via an actuator provided in a touch screen interface on the display unit, wherein a user may cause the intra-oral camera to capture images not by local actuation which may inadvertently cause vibration of the camera housing, but upon independent but wirelessly integrated actuation of a remote actuator.

### BRIEF DESCRIPTION OF THE SEVERAL VIEWS OF THE DRAWINGS

FIG. 1 is a block diagram representing an exemplary embodiment of a wireless intra-oral imaging system according to the present invention.

FIG. 2 is a side image representing an exemplary intra-oral imaging device of the system in FIG. 1.

FIG. 3 is a broken front image representing an exemplary camera module of the device in FIG. 2.

FIG. 4 is an exploded diagram representing an exemplary configuration of the device in FIG. 2.

FIG. 5 is a flowchart representing an exemplary method of operation of the system according to FIG. 1.

### DETAILED DESCRIPTION OF THE INVENTION

Throughout the specification and claims, the following terms take at least the meanings explicitly associated herein, unless the context dictates otherwise. The meanings identified below do not necessarily limit the terms, but merely provide illustrative examples for the terms. The meaning of “a,” “an,” and “the” may include plural references, and the meaning of “in” may include “in” and “on.” The phrase “in one embodiment,” as used herein does not necessarily refer to the same embodiment, although it may.

The term “coupled” means at least either a direct electrical connection between the connected items or an indirect connection through one or more passive or active intermediary devices.

Terms such as “providing,” “processing,” “supplying,” “determining,” “calculating” or the like may refer at least to an action of a computer system, computer program, signal processor, logic or alternative analog or digital electronic device that may be transformative of signals represented as physical quantities, whether automatically or manually initiated.

The term “computer-readable memory medium” as used herein may refer to any non-transitory medium alone or as one of a plurality of non-transitory memory media within which is embodied a computer program product that includes processor-executable software, firmware, or the like which upon execution may provide data or otherwise cause a computer system to implement subject matter or otherwise operate in a specific manner as further defined herein. It may further be understood that more than one type of memory media may be used in combination to conduct processor-executable software, instructions, program modules, etc., from a first memory medium upon which they initially reside to a processor for execution.

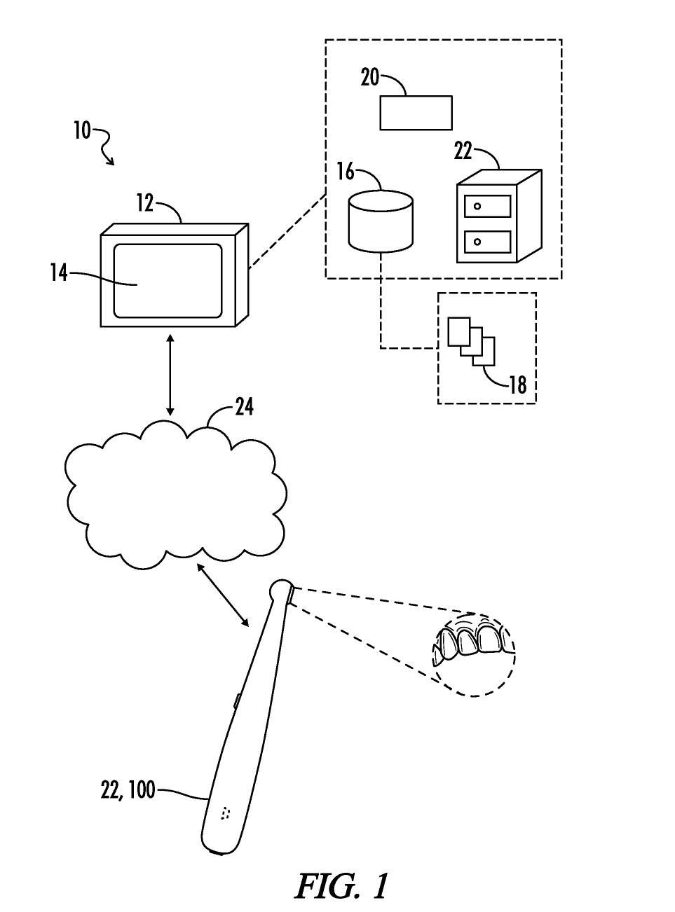

Referring generally to FIGS. 1-5, various embodiments of an intra-cavity imaging system and method may now be described with greater particularity. Where the various figures may describe embodiments sharing various common elements and features with other embodiments, similar elements and features are given the same reference numerals and redundant description thereof may be omitted below.

Referring first to FIG. 1, in an exemplary embodiment the system 10 generally includes a computing device 12 functionally linked to an imaging device 22 via a communications network 24. The term “communications network” as used herein with respect to data communication between at least an imaging device and a remote computing device may refer generally to a BlueTooth protocol and associated components, but may alternatively unless otherwise stated include any one of, or a combination of any two or more of, telecommunications networks (whether wired, wireless, cellular or the like), a global network such as the Internet, local networks, network links, Internet Service Providers (ISP's), and intermediate communication interfaces.

The imaging device 22 as referred to herein may typically be an intra-oral imaging device 22, 100 for use in dental applications, but the device 22 may within the scope of the present invention further refer to imaging devices for use in various alternative applications. For example, in an embodiment the imaging device may be an intra-cavity device used in medical or veterinary applications wherein the healthcare provider may find such intra-cavity images useful or desirable.

In another exemplary embodiment, the imaging device may not be used for intra-cavity applications at all, but may be used by dermatologists or the like for imaging of a patient's skin, hair, etc.

In yet another exemplary embodiment, the imaging device may be used completely outside of the scope of healthcare, such as for example for mechanical device or system imaging as may be utilized by or for mechanical service personnel in identifying and troubleshooting equipment such as for example HVAC systems.

It may be understood that the various embodiments described above may each be implemented without substantial modification to the components, features and steps of the present invention as further described herein, and therefore in other words each of these embodiments and applications may be considered within the scope of the present invention unless otherwise stated or logically required. Where specific steps call for insertion of an intra-oral camera into an oral cavity, for example, it may be understood that equivalent steps can and would be performed in accordance with other embodiments of the present invention to provide analogous results.

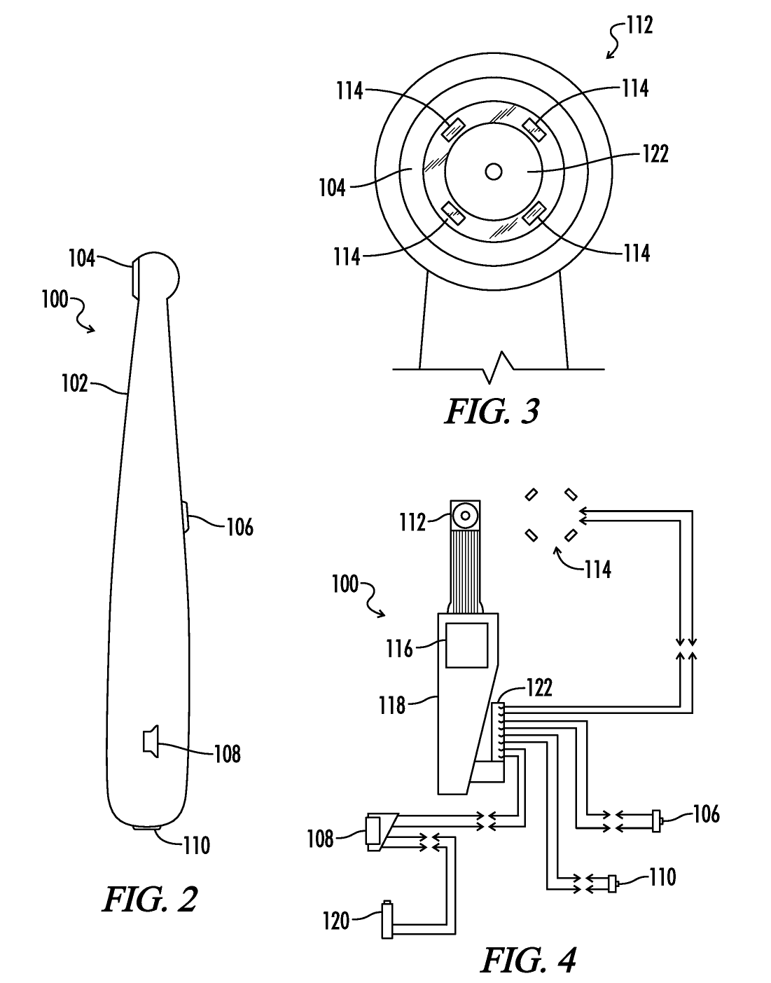

Referring to FIG. 2, in an embodiment the intra-oral imaging device 100 includes a housing 102 which may be portable and preferably capable of being hand-held, but in alternative embodiments the housing may be mounted on for example a jointed arm for more stationary positioning with respect to the mouth of a patient.

The intra-oral imaging device 100 may further include one or more communications ports 108 for data transmission to and from a remote computing device via a communications network. Exemplary communications ports may include USB ports, RF transceivers, data modems or various other equivalent wired and/or wireless data transmission mechanisms within the scope of the present invention.

The camera and device structure may in an embodiment be composed as represented in FIGS. 3-4, but in various embodiments alternative and equivalent configurations may be anticipated within the scope of the present invention.

The intra-oral imaging device may generally be responsive to a touch-sensitive actuator 106 or equivalent user trigger 106 such as for example a button 106 positioned for external manipulation on the housing to utilize an LED array 114 within a camera module 112 to thereby generate image data associated with one or more teeth inside the mouth of a patient. The camera module 112 as represented in FIG. 3 may include for example a camera aperture 122, a clear lens cover and lens trim 104. The camera module 112 may be sized and shaped for positioning on a first end of the imaging device 100 itself wherein the camera module may be inserted into the mouth (or equivalent cavity in other applications) of a user. In certain embodiments (not shown), the device 100 may further include movement sensors such as for example angular velocity sensors wherein program logic may be able to detect and compensate for arbitrary movements during the image capturing process in a manner known in the art. In conjunction with the touch-sensitive actuator, such logic may effectively reduce if not entirely eliminate the vibration effects typical to users' hands.

An exemplary internal configuration of the device 100 as represented in FIG. 3 may include the camera module 112 coupled via a connector such as a ribbon connector 124 to a circuit board 118 upon which resides a processor (not shown) and a computer-readable medium 116 including appropriate program logic. In an embodiment, the medium may be a Secure Digital storage medium as is known in the art, such as for example a high-capacity Micro SD card. In another embodiment, the storage medium and processor may collectively reside in a microcontroller or an equivalent. The circuit board may further include an I/O wiring connector 122 or the equivalent wherein components such as the LED array 114, user actuator 106, power button 110, battery 120 and USB port 108 may be functionally linked to and/or otherwise in electrical communication with the processor.

The image data from the intra-oral imaging device 100 may be transmitted to a portable computing device 12 such as for example a tablet computer or a cell phone which further includes a display interface 14, a processing unit 20, and a computer-readable medium 16 which is programmed to execute, or otherwise includes program instructions 18 associated with, a mobile application that utilizes the image data. The data transmission may, as described above, generally take place in accordance with the present invention via a Bluetooth wireless communications network 24 and associated intermediate components.

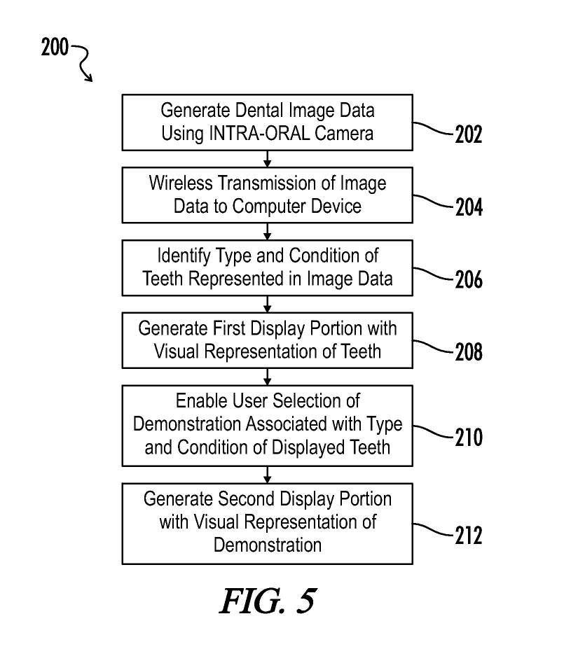

Referring now to FIG. 5, an exemplary method of operation 200 according to embodiments of the present invention may now be described. Such methods may typically incorporate steps carried out by a combination of user actuation of the handheld intra-oral device 100 and also user interaction with a program application executed on the computing device 12, but execution of particular steps in various embodiments may not be strictly limited with respect to one particular device, unless otherwise stated or as inherent to operation of the device itself. The order in which steps take place is not limited to that described herein, again unless otherwise stated or as necessary to perform the steps themselves.

The process begins (step 202) with positioning of the camera module of the imaging device 100 inside of the mouth or an equivalent cavity for a subject, and subsequently capturing images of, e.g., one or more teeth. As mentioned above, the camera may be directed to capture images upon an identified user actuation, which may in an embodiment be merely identifying contact with a touch-sensitive button on the housing of the device 100. In other embodiments, a sequence of events may be identified with respect to for example a touch screen on the display unit of the computing device. The sequence may include as one example identifying a first touch and a subsequent swipe of the finger or equivalent element across the touch screen within a dedicated window associated with camera actuation. The sequence may further include identifying by program logic an approved image capturing state that may be determined in part by signals from one or more angular velocity sensors or the equivalent residing in the imaging device housing.

Upon identifying user actuation, the camera and associated components are utilized to capture a plurality of images. The process by which an intra-oral camera module captures images is known in the art, and may conventionally involve illuminating the subject teeth using the LED array, generating analog signals representative of images of the teeth, and implementing analog-to-digital circuitry to digitally convert the analog signals into digital image data representative of an arrangement and conditions for one or more of the teeth of the subject.

The program logic in the imaging device may be effective to further identify and select images for transmission to the computing device (step 204). The logic may include modules for scoring the images according to clarity, resolution, lighting, etc., wherein unclear images or images that otherwise fall outside of a predetermined threshold are rejected. In an embodiment, only a “best” image is subsequently packaged for transmission to the computing device, and if no images exceed the threshold value the system may prompt the user to retake the relevant images.

As previously mentioned, in an embodiment the computing device may be a mobile device such as a smart phone or tablet computer executing a mobile application which is effective to operate on the received image files and generate one or more dental procedure demonstrations according to the present invention. As a preliminary step, the application may first identify one or more conditions and/or locations of the tooth to facilitate the display and demonstration process (step 206). Where, for example, a sequence of image data files are generated and provided, the system may seek to arrange the sequence of images as a single “panoramic”-style image on the display. Otherwise, the images may be displayed individually and according to for example user selection, such as from a list.

In one example of such a mobile application, the program generates an interface that includes graphical representations of the one or more teeth in substantially real-time for visual aids or demonstration to the associated patient, or otherwise for assistance in a dental procedure or the like (step 208). Such representations may take the form of simple real-time imaging or may be supplemented by for example visual overlays to demonstrate or highlight portions of the teeth or aspects of the teeth which may require present or future attention, or to demonstrate potential procedures and their results with respect to the teeth such as for example an exemplary veneer. While the images may be generated in substantially real-time upon the display, it may be understood that the images may further be saved on the display upon generation, wherein a dental care provider or patient may observe the one or more represented teeth in question after the intra-oral device has been removed.

In another example, the program may begin with a real-time image of the one or more teeth and execute a program module to progressively display a sequence of images culminating with a projected image of equivalent one or more teeth, after a proposed dental procedure has taken place. The program may further be effective to identify criteria such as a shape, size, condition, etc. of the one or more teeth, and further determine an appropriate sequence of images for display based on the identified criteria and further based on predetermined sequences as stored in a database which either resides on the computing device or is communicatively linked to the computing device and accessible during operation of the program.

In another example, or in conjunction with either or both of the above examples, the program may generate a second interface portion which includes textual or graphical demonstrations of a dental procedure associated with the patient or more specifically with the one or more teeth in question. The demonstrations may typically be selectable by either the dental care provider or the patient from a list of demonstrations residing on a database as described above and associated with said computing device (steps 210-212).

In various embodiments, however, certain demonstrations may be automatically selected by the system in view of the current status of one or more teeth, the extrapolated future status of the teeth, and/or historical data for the patient/subject. The program may obtain such historical data associated with the subject from a local or remote database that is accessible by the computing device for comparison against the current images of the teeth, and/or extrapolated future conditions of the teeth. The comparisons may typically aid system determinations as to which procedure and/or demonstration would optimally be displayed to the users. For example, if a current condition of a particular tooth is consistent with a historical condition of the same tooth, that would imply somewhat different circumstances than where for example the condition of the tooth had deteriorated rapidly over the same time frame. Further, relative collective arrangement and conditions for the entire set of teeth by way of comparison over time may presumably influence the extrapolation of future conditions and appropriate forecasting of dental procedures that may be required based on those conditions.

In various embodiments the demonstrations may be either selected automatically by the system or may be selected by a user from a list, that list being automatically generated by the system as relevant to the subject based upon analysis of any or all of the historical teeth data, current teeth data and extrapolated or projected future conditions of the teeth.

Projections moving forward with respect to the teeth of the subject may often be best provided by a dentist user, particularly where there is insufficient historical data or other input data sufficient to allow for relative analysis. Embodiments of the present invention may utilize sophisticated program engines such as machine learning algorithms to improve the analytics and forecasting capabilities over time, but such engines are inherently weakest when the amount of accessible data is sparse, as for example with a first-time patient. Therefore, in various embodiments of the present invention the system may rely on a sliding scale of tools ranging from full user input at a first stage of the extrapolation process for a given user, to a combination of user input and rules-based analysis as data is gathered (which may be specific to the user and/or relevant to the user based on analogous cases on file), and finally to a predominantly automated and data-driven analysis.

The previous detailed description has been provided for the purposes of illustration and description. Thus, although there have been described particular embodiments of the present invention of a new and useful “wireless intra-oral imaging system and method,” it is not intended that such references be construed as limitations upon the scope of this invention except as set forth in the following claims.

What is claimed is:

1. An intra-oral imaging method comprising:
causing an intra-oral camera in response to a user trigger to capture video images of a set of one or more teeth of a subject user;
generating image data representative of an arrangement and conditions for the set of teeth;
transmitting the image data from the intra-oral camera to a computing device via a wireless communication network;
generating graphical representations of the set of teeth on a display unit functionally linked to the computing device;
identifying a current condition of one or more of the teeth in the set of teeth;
selecting a prerecorded visual demonstration of a dental procedure from a database associated with the computing device, based on the identified current condition of the one or more teeth; and
displaying the demonstration of the dental procedure on the display unit.

2. The method of claim 1, said user trigger comprising a predefined user interaction with a touch-sensitive actuator coupled to the camera.

3. The method of claim 1, said user trigger comprising completion of an identified sequence further comprising user engagement with a touch screen interface associated with the display unit.

4. The method of claim 1, further comprising steps of:
comparing the current condition of the one or more teeth to respective historical data associated with the teeth; and
determining one or more instances of recommended actions based on the current condition and the respective historical data for the teeth,
wherein the step of selecting a prerecorded visual demonstration based on the identified current condition of the one or more teeth further comprises selecting one or more prerecorded visual demonstrations based on the recommended actions.

5. The method of claim 4, further comprising a step of dynamically generating and displaying on the display unit a visual sequence representative of future conditions of one or more teeth as extrapolated from the current condition and the historical data associated with the respective one or more teeth.

6. The method of claim 5, wherein selecting one or more prerecorded visual demonstrations based on the recommended actions comprises selecting one or more prerecorded visual demonstrations of a potential dental procedure associated with the extrapolated future condition of one or more teeth.

7. The method of claim 4, wherein selecting one or more prerecorded visual demonstrations based on the recommended actions comprises selecting one or more prerecorded visual demonstrations of a recommended dental procedure associated with the current condition of one or more teeth.

8. The method of claim 7, wherein the one or more prerecorded visual demonstrations comprise one or more selectable colors and shapes associated with a potential dental procedure, said selectable colors and shapes generated based on a cumulative condition of the set of teeth.

9. An intra-oral imaging system comprising:
a handheld imaging device further comprising a housing within which resides a touch-sensitive actuator, an intra-oral camera, and a first non-transitory computer-readable storage medium, the first medium further comprising program instructions executable by a processor to direct the performance of:
causing the intra-oral camera in response to actuation of the touch-sensitive actuator to capture images of one or more teeth of a subject user,
generating image data based on the captured images, the image data representative of an arrangement and conditions for the one or more teeth,
transmitting the image data via a wireless communications network; and
a computing device further comprising a display unit and a second non-transitory computer-readable storage medium, the second medium further comprising program instructions executable by a processor to direct the performance of:
receiving the image data from the imaging device via the wireless communication network,
generating graphical representations of the teeth on the display unit,
identifying a current condition of one or more of the teeth,
selecting a prerecorded visual demonstration of a dental procedure from a database, based on the identified current condition of the one or more teeth, and
displaying the demonstration of the dental procedure on the display unit.

10. The system of claim 9, said actuator comprising a touch-sensitive button coupled to the camera.

11. The system of claim 9, the program instructions in the second medium further executable to direct the performance of:
comparing the current condition of the one or more teeth to respective historical data associated with the teeth; and
determining one or more instances of recommended actions based on the current condition and the respective historical data for the teeth,
wherein selecting a prerecorded visual demonstration based on the identified current condition of the one or more teeth further comprises selecting one or more prerecorded visual demonstrations based on the recommended actions.

12. The system of claim 11, the program instructions in the second medium further executable to direct the performance of dynamically generating and displaying on the display unit a visual sequence representative of future conditions of one or more teeth as extrapolated from the current condition and the historical data associated with the respective one or more teeth.

13. The system of claim 12, wherein selecting one or more prerecorded visual demonstrations based on the recommended actions comprises selecting one or more prerecorded visual demonstrations of a potential dental procedure associated with the extrapolated future condition of one or more teeth.

14. The system of claim 11, wherein selecting one or more prerecorded visual demonstrations based on the recommended actions comprises selecting one or more prerecorded visual demonstrations of a recommended dental procedure associated with the current condition of one or more teeth.

15. The system of claim 14, wherein the one or more prerecorded visual demonstrations comprise one or more selectable colors and shapes associated with a potential dental procedure, said selectable colors and shapes generated based on a cumulative condition of the set of teeth.

16. A non-transitory computer-readable storage medium comprising program instructions executable by a processor to direct the performance of:
identifying a predefined user actuation sequence of a user actuator;
causing an intra-oral camera in response to the user actuation sequence to capture video images of a set of one or more teeth of a subject user;
generating image data representative of an arrangement and conditions for the set of teeth;
transmitting the image data from the intra-oral camera to a remote computing device via a wireless communication network;
generating graphical representations of the set of teeth on a display unit functionally linked to the computing device;
identifying a current condition of one or more of the teeth in the set of teeth;
selecting a prerecorded visual demonstration of a dental procedure from a database associated with the computing device, based on the identified current condition of the one or more teeth; and
displaying the demonstration of the dental procedure on the display unit.

17. The medium of claim 16, said predefined user actuation sequence of a user actuator comprising a user manipulation of a touch-sensitive actuator electrically coupled to the camera.

18. The medium of claim 16, said predefined user actuation sequence of a user actuator comprising completion of an identified sequence of steps wherein a user engages a touch screen interface associated with the display unit.

19. The medium of claim 16, the program instructions further executable by the processor to direct the performance of:
comparing the current condition of the one or more teeth to respective historical data associated with the teeth; and
determining one or more instances of recommended actions based on the current condition and the respective historical data for the teeth,
wherein the step of selecting a prerecorded visual demonstration based on the identified current condition of the one or more teeth further comprises selecting one or more prerecorded visual demonstrations based on the recommended actions.

20. The medium of claim 19, further comprising a step of dynamically generating and displaying on the display unit a visual sequence representative of future conditions of one or more teeth as extrapolated from the current condition and the historical data associated with the respective one or more teeth,
wherein selecting one or more prerecorded visual demonstrations based on the recommended actions comprises selecting one or more prerecorded visual demonstrations of a potential dental procedure associated with the extrapolated future condition of one or more teeth.
